(19) United States
(12) Patent Application Publication
(10) Pub. No.: US 2026/0070853 A1
(43) Pub. Date: Mar. 12, 2026

(54) PROCESS FOR PRODUCING BIOFERTILIZERS/BIOSTIMULANTS FROM MICROALGAE OF THE SPECIES SCENEDESMUS SP. WITH INTENSIVE CO2 CAPTURE, BIOFERTILIZERS/BIOSTIMULANTS OBTAINED BY SAID PROCESS AND THEIR USES IN AGRICULTURE

(71) Applicants: PETRÓLEO BRASILEIRO S.A. - PETROBRAS, Rio de Janeiro (BR); UNIVERSIDADE FEDERAL DO RIO DE JANEIRO — UFRJ, Rio de Janeiro (BR)

(72) Inventors: LEONARDO BRANTES BACELLAR MENDES, Rio de Janeiro (BR); ALANE BEATRIZ VERMELHO, Rio de Janeiro (BR); LIVIA MARQUES CASANOVA, Rio de Janeiro (BR); ANDREW MACRAE, Rio de Janeiro (BR); RONALDO BERNARDO DA SILVA, Rio de Janeiro (BR); HERALDO NAMORATO DE SOUZA, Rio de Janeiro (BR); RAFAEL RICHARD JOAO, Rio de Janeiro (BR)

(73) Assignees: PETRÓLEO BRASILEIRO S.A. - PETROBRAS, Rio de Janeiro (BR); UNIVERSIDADE FEDERAL DO RIO DE JANEIRO — UFRJ, Rio de Janeiro (BR)

(21) Appl. No.: 19/320,343

(22) Filed: Sep. 5, 2025

(30) Foreign Application Priority Data
Sep. 12, 2024 (BR) 1020240187660

(51) Int. Cl. C05F 11/08; A01G 22/00; A01G 24/20; C05F 17/20; C12N 1/12; C12R 1/89; G01N 30/02; G01N 30/88

(52) U.S. Cl. CPC C05F 11/08; A01G 22/00; A01G 24/20; C05F 17/20; C12N 1/125; G01N 30/88; C12R 2001/89; G01N 2030/027; G01N 2030/8818; G01N 2030/8836; G01N 2030/884

(57) ABSTRACT

The present invention relates to a process for producing biofertilizers/biostimulants from microalgae with intensive CO₂ capture, which comprises the following steps:
a) preparing an inoculum;
b) cultivating for the production of microalgae biomass in a window photobioreactor;
c) collecting the biomass by flocculation;
d) drying the biomass, and grinding and extracting a polar fraction by solvent in a two-phase system; and
e) analyzing the polar fraction by liquid chromatography.

The invention also relates to the extracts of wet biomass of the microalgae Scenedesmus sp., obtaining the polar fraction. This polar fraction contains amino acids, sugars, betaine derivatives, vitamin B3 (nicotinamide or nicotinic acid) and pantothenic acid (vitamin B5), the monoterpene loliolide and the tryptophan derivative indole-lactic acid (analogous to indole-acetic acid, a phytohormone of the auxin class).

The polar fraction can also be used as an input in the cultivation of soybeans, corn, sugar cane, cotton and coffee, among other cultivated species.

### CROSS-REFERENCE FOR RELATED APPLICATIONS

This application claims priority to Brazilian Application No. BR 1020240187660, filed on Sep. 12, 2024, the disclosure of which is herein incorporated by reference in the entirety.

### FIELD OF THE INVENTION

The present invention has as its field of application the area of sustainable development. More specifically, the present invention relates to a process for producing biofertilizers/biostimulants from microalgae with intensive CO: capture and their use as natural biofertilizers and biostimulants in agriculture.

### BACKGROUND OF THE INVENTION

CO₂ emissions from “carbon-intensive” industries, such as those found in refineries and thermoelectric plants, have a major impact on climate change, since CO: is one of the main gases responsible for global warming and its emissions in large quantities contribute to the increase in the greenhouse effect.

Companies seeking environmental sustainability are looking for solutions to mitigate their emissions through “post-combustion” strategies, with most “direct” CO₂ fixation technologies having worse cost/benefit ratios when compared to “indirect” options such as CO₂ biofixation via the biological route.

Microalgae cultivation has the potential to contribute to the reduction and neutralization of CO₂ emissions and can expand the supply of non-energy products that are resilient to the energy transition.

Microalgae are efficient biological CO₂ sequestrators, contributing to the reduction of CO₂ levels and increasing atmospheric O₂ concentrations through photosynthesis. Microalgae are capable of removing CO₂ from gaseous emissions from various sources, supporting CO₂ concentrations of up to 50% and the presence of other gases such as NOx and CO₂. Compared to trees, which have an estimated capacity to fix CO₂ of 1 t·ha⁻¹·year⁻¹ to 3.5 t·ha⁻¹·year⁻¹ in tropical forests, microalgae can fix 6.3 t·ha⁻¹·year⁻¹, reaching 16.2 t·ha⁻¹·year⁻¹ in tropical regions of Brazil with high insolation. Their rapid growth and high tolerance to various extreme abiotic factors allow them to be cultivated in cycles of just a few days, in wastewater of various origins, saline or unsuitable for other human use or in agriculture, and they can be cultivated on non-arable land, not competing with food production.

The decarbonization process is aligned with the energy transition and seeks to replace fossil fuel-based energy sources with renewable sources of liquid fuels, electricity and hydrogen.

The capture, storage and use of carbon are crucial to guarantee the valorization of bioenergy, bioproducts and bioinputs, such as biofertilizers/biostimulants, a natural product, contributing to environmental sustainability and the circular bioeconomy.

Biofertilizers and biostimulants are circular because they are biologically based, promote the recycling of nutrients and can improve the chemical, physical and biological attributes of the soil.

Nutrient recycling can be carried out, for example, by using drilling gravel waste generated in oil and gas exploration and production activities as a mineral nutrient substrate for the cultivation of microalgae, as described in patent applications BR 102021026904-9 and BR 102022006198-0, both from the same authors of this patent application.

The patent application BR 102021026904-9 proposes the use of environmental liabilities consisting of solid waste (drilling gravel) from drilling activities coupled with CO₂ biofixation using cyanobacteria. Cyanobacteria fix atmospheric nitrogen in their biomass, enriched with nitrogen obtained biologically, without the high energy expenditure typical of traditional synthetic nitrogen fertilizers, to obtain organomineral fertilizer, in such a way that there is added value to an environmental liability generated by oil and gas exploration and production (E&P) activities. The resulting organomineral fertilizer has mechanisms that stimulate the tolerance of fertilized plants to salinity, sodicity and possible contamination of petroleum hydrocarbons present in the drilling gravel used as a mineral matrix in the formulation of this organomineral fertilizer.

The patent application BR 102022006198-0 also deals with the direct use of drilling gravel residue from oil exploration and production activities in the formulation of culture media for unicellular organisms, increasing their growth rates, intensifying CO₂ biofixation, and generating value for this residue from the productivity gains expected by autotrophic and mixotrophic organisms (cyanobacteria, microalgae and macroalgae), as well as by the production of bioproducts that can be generated through the biorefining concept. Algae are cultivated using drilling gravel suspended in the culture medium together with the ability to grow by absorbing CO₂. The mechanisms used by algae in soils and marine environments to tolerate salinity, sodicity and contamination by petroleum hydrocarbons provide broad adaptation to these abiotic stress conditions and enable disposal without environmental impact, constituting a satisfactory solution for the disposal of gravel for oil exploration and production.

The patent BR PI 1107211-3 of the Federal University of Paraná (UFPR) already focuses on biomass production and describes a process for cultivating biomass on a laboratory scale, separating it, and extracting substances of interest. The microalgae used in the process are Scenedesmus sp., grown in photobioreactors at temperatures between 18 and 20° C. and in modified Guillard F/2 culture medium, with the biomass separated from the culture medium through flocculation, centrifugation, and filtration, followed by freezing and dehydration by drying, lyophilization, etc. The dehydrated biomass is subjected to sequential extractions with solvents in order of polarity (hexane, ethanol, and water), and the extracts were used in agronomic experiments. The document also describes the technique for using extracts and suspensions of the microalgae Scenedesmus sp. in seeds, localized irrigation, and spraying of the aerial part.

The present invention describes a process for cultivating biomass on an industrial scale, separating it and extracting the substances of interest. The microalgae used in the process are Scenedesmus sp. with thermophilic characteristics, cultivated in photobioreactors at temperatures between 4° and 50° C. and in a modified BG-11 culture medium, with the biomass being separated from the culture medium only by flocculation, followed by drying and grinding. The dried and ground biomass is subjected to solvent extraction in a two-phase system, and the extract containing the polar fraction is analyzed by liquid chromatography. The polar fraction of the biomass produced is a basic liquid formulation, ready for direct application by the farmer, only making the appropriate dilutions.

Currently, the use of biofertilizers/biostimulants has received increasing attention as an approach to stimulate plant growth and productivity in a sustainable manner. These compounds improve the efficiency of mineral nutrition through the distribution of nutrients within the plant, tolerance to abiotic stresses (salinity, drought, high and low temperatures, heavy metals, etc.) and biotic stresses (pests, diseases, invasive plants, etc.), stimulate vegetative growth, increase the antioxidant capacity, vigor and yield of agricultural crops, and improve product quality characteristics, regardless of their content in essential mineral nutrients for plants in agricultural production systems. This action may involve direct mechanisms acting on the physiology and metabolism of plants or indirect mechanisms, acting on the conditions of the soil (rhizosphere) and the microbiota associated with it.

Most patent applications and granted patents use extracts of total biomass. In the case of the present invention, it is proposed to use only polar substances (polar fraction), obtained from biomass extraction processes, after the removal of lipids with nonpolar solvents. Such lipids can be used for biodiesel production, while the remaining polar substances can be used as biofertilizers/biostimulants for use in agriculture, since they are high-value inputs with high and growing demand in agriculture. In this way, it would be possible to integrate biodiesel production with biofertilizer/biostimulant production, allowing for cost and waste generation reductions.

Therefore, the present invention has the potential to overcome current processes of direct use of CO₂, without net emissions, since it provides better use compared to carbon capture for storage (CCS) through sustainable production of biofertilizer/biostimulant.

The present invention also has the potential to use and recycle drilling cuttings, which are solid waste generated by oil and gas exploration and production activities, as a mineral nutrient substrate for cultivation. This waste, due to its quantity and high salinity, requires environmentally appropriate treatment and disposal in accordance with current environmental standards and requires overcoming logistical, technical and economic challenges.

The present invention also has fewer associated risks and difficulties because it does not involve the intensive extraction of natural resources from conventional raw materials and the use of environmentally damaging chemical agents used in the current traditional routes for producing biofertilizers/biostimulants.

### SUMMARY OF THE INVENTION

The present invention relates to a process for producing biofertilizers/biostimulants from microalgae with intensive CO₂ capture, which comprises the following steps:
a) preparing the inoculum;
b) cultivating for the production of microalgae biomass in the window photobioreactor;
c) collecting the biomass by flocculation;
d) drying the biomass, grinding and extracting the polar fraction by solvent in a two-phase system; and
e) analyzing the polar fraction by liquid chromatography.

The invention also relates to the polar fraction extracted from the biomass of the microalgae Scenedesmus sp. This polar fraction contains amino acids, sugars, betaine derivatives, vitamin B3 (nicotinamide or nicotinic acid) and pantothenic acid (vitamin B5), the monoterpene loliolide and the tryptophan derivative indole-lactic acid (analog of indole-acetic acid, a phytohormone of the auxin class).

The polar fraction can also be used in crops of agricultural and economic importance and with greater demands for fertilizer consumption in Brazil, such as soybeans, corn, sugarcane, cotton and coffee, among other cultivated species. It has the potential to optimize or replace the use of fertilizers.

### BRIEF DESCRIPTION OF THE DRAWINGS

FIGS. 1A, 1B, 1C, 1D, 1E, 1F, 1G and 1H show the cultivation and evaluation of the action of the biostimulant in plants of the Setaria viridis species.

FIG. 1A shows the seedlings 5 days after germination.

FIG. 1B shows the plants transferred to the hydroponic system.

FIGS. 1C, 1D and 1E show the plants 15 days, 28 days and 77 days after the start of the treatments, respectively.

FIG. 1F shows the panicle bagged for seed collection.

FIG. 1G shows the seeds.

FIG. 1H shows the roots.

FIG. 2 shows the stages of biomass extraction of Scenedesmus sp. in a two-phase system.

FIGS. 3A, 3B, 3C and 3D are graphs of the effects of treatments with Scenedesmus sp. on biometric parameters of Setaria viridis after 11 weeks of experiment. The biometric parameters are the dry weight of aerial parts (FIG. 3A), the dry weight of root (FIG. 3B), the height (FIG. 3C), and the length of panicles (FIG. 3D).

### DETAILED DESCRIPTION OF THE INVENTION

To obtain microalgae biomass, it is necessary to add the culture medium and the inoculum (microalgae strain) to the cultivation system. CO₂ is inserted into the system at a flow rate of 5 l/min to control the pH when it reaches a value of 6.5. From the nutrients provided and injected CO₂, the strain used grows over the days of cultivation. After cultivation, the microalgae biomass is collected by flocculation. The collected wet biomass is dried in an oven for 48 h at 60° C. and crushed for subsequent extraction with solvents. In the extraction step, three 200 mg aliquots were used. To each aliquot, 2.5 ml of methanol/water (1:1) was added. The material was subjected to ultrasound for 20 minutes and then 2.5 ml of chloroform was added. After another 10 minutes of sonication, the samples were centrifuged at 5.000 rpm for 20 minutes, forming two phases that are collected separately: the aqueous phase or polar fraction containing methanol/water and the organic phase or nonpolar fraction containing chloroform. The nonpolar fraction can be used for biodiesel production, while the polar fraction is further analyzed by liquid chromatography to be used as biofertilizers/biostimulants in agriculture.

The polar fraction contains amino acids, sugars, betaine derivatives, vitamin B3 (nicotinamide or nicotinic acid) and pantothenic acid (vitamin B5), the monoterpene loliolide and the tryptophan derivative indole-lactic acid (analog of indole-acetic acid, a phytohormone of the auxin class).

The polar fraction generated is the product that will be marketed as a biofertilizer/biostimulant.

The successful use of microalgae as a biofertilizer/biostimulant depends on many factors, such as the species of microalgae cultivated, the cultivation method, biomass processing, inoculum concentration, application dosage, and physical nature of the product (fluid or solid).

In the cultivation stage of the present invention, a strain of microalgae Scenedesmus sp. with thermophilic characteristics was used, capable of growing at extreme temperatures, around 50° C. The cultivation was conducted in window photobioreactors with a volume of 100 liters and with a cultivation temperature ranging from 40° C. to 50° C. The cultivation medium used was modified BG-11 in order to reduce costs, without affecting the biomass productivity of the cultivation. The modified BG-11 medium has urea as its nitrogen source. In the standard BG-11 medium, sodium nitrate is used instead of urea. In addition, in the modified BG-11 medium, the trace metal solution was removed from the medium.

Table 1 below shows the composition of the modified BG-11 medium:

| Components | Amounts | |
|---|---|---|
| 1. FeCl₃•6H₂O | 0.2 to 0.8 | mg/l |
| 2. Ureia | 20 to 80 | mg/l |
| 3. K₂HPO₄ | 10 to 30 | mg/l |
| 4. MgSO₄•7H₂O | 5 to 20 | mg/l |
| 5. NaCl | 5 to 35 | g/l |

In the flocculation step of the present invention, a special organic polyamide flocculant (polyelectrolyte) at a concentration of 3 mg/l was used to separate the supernatant from the concentrated wet biomass in order to reduce the cost of the collection step.

The polar fraction of the biomass produced in the present invention (obtained at each cultivation cycle) can be used as biofertilizers and/or biostimulants directly, that is, it can be considered as a basic liquid formulation, ready for direct application by the farmer by making the appropriate dilutions.

Other components based on traditional mineral fertilizers with high water solubility, such as urea, phosphoric acid, potassium chloride and other soluble fertilizers that provide N, P, K, S, Ca, Mg, B, Cu, Zn, Mn, Cl, Mo, Fe and other plant nutrients, can be added to this basic liquid formulation, and liquid organomineral fertilizers with additional biofertilizer and biostimulant effects can be formulated.

This same basic liquid formulation can be subjected to drying processes, for example, via a rotary drum dryer, and concentration, thus obtaining a dry organic matrix with lower moisture content (up to 20% weight/weight). Other traditional solid mineral fertilizers such as urea, monoammonium phosphate, diammonium phosphate, potassium chloride and other solid fertilizers that supply N, P, K, S, Ca, Mg, B, Cu, Zn, Mn, Cl, Mo, Fe and other mineral nutrients can be added to this dry organic matrix, and solid organomineral fertilizers with additional biofertilizer and biostimulant effects can be formulated.

### Embodiments of the Invention and Results Obtained

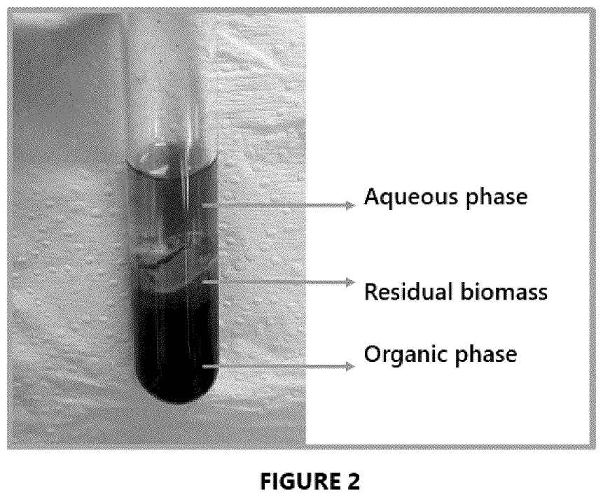

The microalgae Scenedesmus sp. was cultivated in a closed photobioreactor using modified BG-11 medium and solar lighting. The system includes bubbling for aeration and CO₂ injection for pH control. Then, the biomass is collected by flocculation. The wet biomass of the microalgae is dried in an oven and crushed. After grinding, the biomass is subjected to an extraction process using a two-phase system, generating an aqueous phase, an organic phase and residual biomass, as shown in FIG. 2. The aqueous and organic phases were then collected separately: methanol/water r (polar fraction-aqueous phase) and chloroform (nonpolar fraction-organic phase). After extraction, analyses are performed by high-performance liquid chromatography coupled to sequential mass spectrometry (HPLC-MS/MS) to identify the substances present in the polar fraction and evaluate their biofertilizer/biostimulant activity.

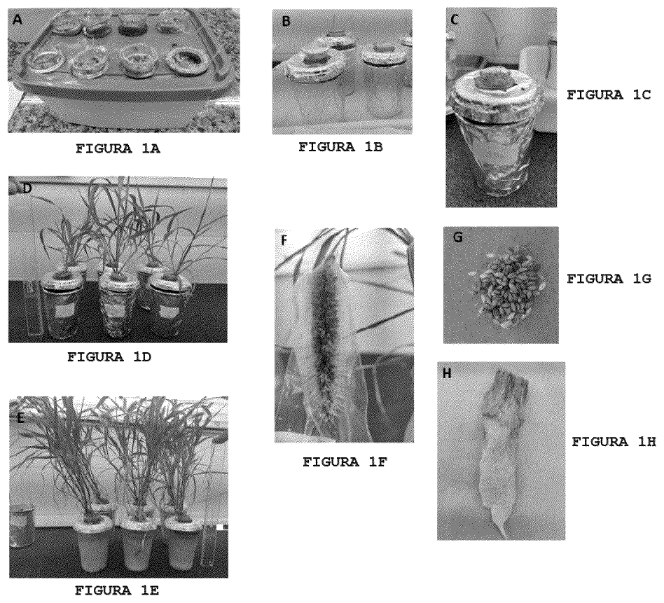

For the evaluation, plants of the species Setaria viridis grown in a hydroponic system using a 25% Hoagland nutrient solution were used. When the plants reached 15 days of age, treatment with the polar fraction of the microalgae was started. A stock solution of the polar fraction at 5 mg/ml was diluted in 25% Hoagland solution to obtain two treatment solutions: D1 (300 mg/l) and D2 (50 mg/l). The plants were divided into 3 groups (n=6): Control (only Hoagland solution), solution D1 and solution D2. The solutions were replaced weekly. The plants were treated for 7 weeks, until the beginning of the reproductive phase. After this period, the treatment was interrupted, and the plants were maintained only with Hoagland solution for another 4 weeks. Finally, the plants were removed from the hydroponic system and measured. The aerial parts were separated from the roots, and both were dried at a temperature of 60 to 70° C. to determine the dry weight. The steps of the experiment are shown in FIGS. 1A to 1H.

### Biomass Extraction

The biomass of Scenedesmus sp. was extracted using a two-phase system, using water/methanol 1:1 for the aqueous phase and chloroform for the organic phase, as shown in FIG. 2. In the polar fraction, amino acids, sugars and betaine derivatives were identified (Table 2). In addition, two vitamins were detected: B3 and B5. Another substance that deserves to be highlighted is loliolide, a monoterpene common in plants and microalgae. The tryptophan derivative, indole-lactic acid, also stands out. This substance corresponds to an analogue of indole acetic acid (IAA), a phytohormone of the auxin class, found in plants and algae, which participates in the regulation of growth and nutrient uptake, among other activities. Many of the substances detected in this fraction can act as biofertilizers/biostimulants (betaines and vitamins, as well as indole lactic acid).

Table 2 below shows the chromatographic and spectrometric properties of the substances identified in the polar fractions of Scenedesmus sp. biomass by HPLC-MS/MS in positive mode.

| Tr | | | | | |
|---|---|---|---|---|---|
| (min) | m/Z | Ion | Main fragments | Structural proposal | Chemical class |
| 2, 74 | 227, 12772 | M + H | 163; 131; 94 | Oxododecanoic Acid | Fatty acid |
| | | | (extensive | | |
| | | | fragmentation) | | |
| 7, 49 | 236, 1524 | M + H | 74; 59; 58; 57 | Betaine derivative | Quaternary |
| | | | | (lipid part) | amine |
| 7, 46 | 237, 1531 | M + 2H | 236; 144; 60; | Betaine derivative | Quaternary |
| | | | 59; 58; 57 | (lipid part) | amine |
| 9, 22 | 258, 1122 | M + H | 184; 125; 104; | Glycerophospho | Quaternary |
| | | | 86 | choline | amine |
| 7, 49 | 471, 2927 | M + H | 236 | Betaine derivative | Quaternary |
| | | | | (dimer) | amine |
| 5, 95 | 104, 1098 | M | 60; 58; 45 | Choline | Quaternary |
| | | | | | amine |
| | | | | | (nutrient) |
| 7, 34 | 162, 1136 | M + H | 58; 59 | Choline derivative | Quaternary |
| | | | | | amine |
| | | | | | (nutrient) |
| 7, 55 | 116, 0717 | M + H | 71; 70 | Proline | Amino acid |
| 6, 60 | 132, 1024 | M + H | 86; 69; 44 | Isoleucine/leucine | Amino acid |
| 12, 31 | 133, 098 | M + H | 116; 70 | Ornithine | Amino acid |
| 5, 82 | 146, 1188 | M + H | 100; 69; 58; 44 | Methylisoleucine | Amino acid |
| 11, 98 | 147, 1134 | M + H | 130; 84 | Lysine | Amino acid |
| 10, 41 | 148, 061 | M + H | 130; 102; 84; | Glutamic acid | Amino acid |
| | | | 56 | | |
| 6, 46 | 166, 0868 | M + H | 121; 120; 53 | Phenylalanine | Amino acid |
| 11, 80 | 175, 1199 | M + H | 175; 158; 130; | Arginine | Amino acid |
| | | | 116; 70; 60 | | |
| 9, 83 | 176, 1034 | M + H | 159; 141; 116; | Citrulline | Amino acid |
| | | | 115; 114; 113; | | |
| | | | 70 | | |
| 7, 82 | 182, 0814 | M + H | 165; 147; 136; | Tyrosine | Amino acid |
| | | | 123; 119 | | |
| 10, 10 | 203, 1507 | M + H | 172; 158; 133; | Dimethyl-arginine | Amino acid |
| | | | 116; 89; 71; 80 | | |
| 24, 82 | 205, 0881 | M + H | 188; 170; 159; | Tryptophane | Amino acid |
| | | | 146; 144; 132; | | |
| | | | 130; 118 | | |
| 3, 64 | 126, 056 | M + H—H₂O | 108; 80; 53 | Pyrohomoglutamic | Amino acid |
| | | | | acid | (derivative) |
| 5, 66 | 278, 16 | M + H | 260; 236; 149; | Deoxyhexosyl-leucine | Glycosidic |
| | | | 117; 58 | | amino acid |
| 8, 51 | 280, 1392 | M + H | 262; 198; 149; | Hexosyl-valine | Glycosidic |
| | | | 130; 72 | | amino acid |
| 7, 96 | 294, 1545 | M + H | 276; 258; 230; | Hexosyl-isoleucine | Glycosidic |
| | | | 212; 161; 144; | | amino acid |
| | | | 132 | | |
| 12, 92 | 295, 1508 | M + H | 277; 272; 263; | Hexosyl-ornitine | Glycosidic |
| | | | 175; 158; 130 | | amino acid |
| 12, 75 | 309, 1662 | M + H | 291; 273; 225; | Hexosyl-lisine | Glycosidic |
| | | | 130; 128 | | amino acid |
| 12, 48 | 337, 1718 | M + H | 320; 302; 175; | Hexosyl-arginine | Glycosidic |
| | | | 158; (extensive | | amino acid |
| | | | fragmentation) | | |
| 7, 87 | 255, 1081 | M + H | 239; 185; 164; | Hexosamine | Amino sugar |
| | | | 145; 127; 85; | derivative | |
| | | | 61 | | |
| 7, 03 | 180, 0872 | M + H | 162; 152; 147; | Hexosamine | Amino sugar |
| | | | 144; 134; 133 | | |
| 1, 94 | 274, 2743 | M + H | 256; 230; 212; | Lauryl | Amino alcohol |
| | | | 106; 102; 88; | diethanolamine | |
| | | | 71; 72; 58 | | |
| 1, 92 | 302, 3054 | M + H | 284; 258; 240; | Tetradecyl | Amino alcohol |
| | | | 106; 88; 70; 57 | diethanolamine | |
| 7, 02 | 198, 0973 | M + NH₄ | 163; 145; 127; | Hexose | Carbohydrate |
| | | | 85; 55 | | |
| 8, 88 | 365, 1064 | M + Na | 203; 185 | Dihexoside | Carbohydrate |
| 5, 26 | 137, 0466 | M + H | 119; 110; 55 | Hypoxanthine | Purine |
| 6, 44 | 152, 0572 | M + H | 135; 128; 110 | Guanine | Purine |
| 2, 99 | 282, 1197 | M + H | 136 | Methyladenosine | Purine |
| 1, 04 | 197, 1198 | M + H | 179; 161; 135; | Loliolide | Terpenoid |
| | | | 133 | (monoterpene) | |
| 1, 06 | 432, 2384 | M + H | 281; 147; 135; | Phorbol (diterpene) | Terpenoid |
| | | | 119 | derivative | |
| 5, 86 | 139, 0512 | M + H | 121; 93 | Nicotinamide | Vitamin B3 |
| | | | | (derivative) | |
| 5, 35 | 220, 1186 | M + H | 202; 184; 174; | Pantothenic acid | Vitamin B5 |
| | | | 160; 142; 90; | | |
| | | | 85; 57 | | |
| 6, 94 | 188, 0708 | M + H—H₂O | 170; 146; 144; | Indole lactic acid | Tryptophan |
| | | | 143; 142; 118; | | metabolite |
| | | | 115 | | |
| 7, 93 | 305, 1343 | M + H | 287; 269; 227; | Deoxyfructosazine | Pyrazine |
| | | | 209; 191; 173; | | |
| | | | 162; 149 | | |

*Tr = Retention time;
*m/Z = mass/charge ratio of ions;

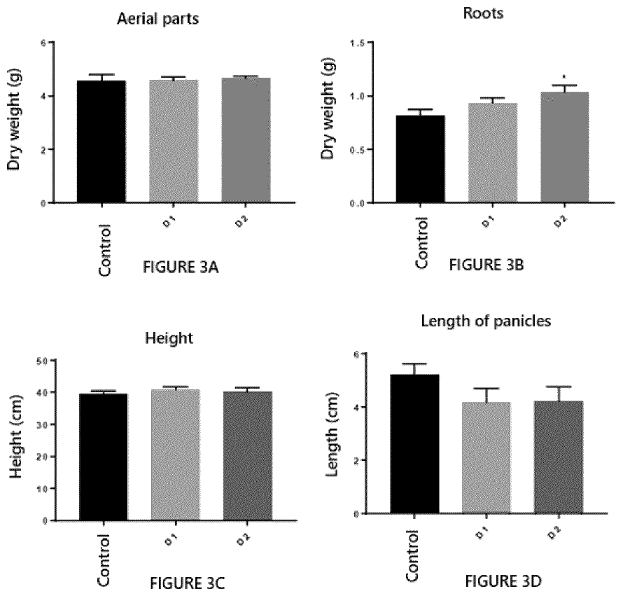

The plants in the three groups (Control, D1 and D2) did not show differences in height (FIG. 3C). The dry weight of the aerial parts was also similar (FIG. 3A). On the other hand, differences were observed in the dry weight of the roots (FIG. 3B), in the average size of the panicles (FIG. 3D) and in the number of seeds. The roots of the plants in the treated groups (D1 and D2) showed greater mass (15% and 27% increase compared to the control, respectively). Regarding the seeds, in the control group, an average of 501 seeds per individual was observed, while D1 and D2 showed averages of 254 and 226, respectively. The difference in the number of seeds may be related to the degree of maturation of the plants.

The results of the study show that the polar extract of Scenedesmus sp. was able to generate physiological changes in the plants evaluated, influencing root development.

The results obtained with the basic liquid formulation demonstrated a direct positive effect on the root development of the Setaria viridis species. The accelerated root development proven at the experimental level in this invention offers several benefits to cultivated plants, positively impacting both their growth and general development of the entire plant, providing greater productivity, as well as plant health, providing greater protection against biotic stresses such as pests, diseases, invasive plants, etc., in addition to protection against abiotic stresses such as drought or excess humidity, high or low temperatures, high or low sunlight, etc. With this result, it is possible to extend this same effect to other species of cultivated plants of agricultural and economic interest.

1. A process for producing biofertilizers/biostimulants from microalgae with intensive CO₂ capture, comprising the following steps:
a) preparing an inoculum;
b) cultivating for the production of microalgae biomass in a window photobioreactor at temperatures between 40° C. and 50° C.;
c) collecting the biomass by flocculation;
d) drying the biomass, and grinding and extracting a polar fraction by solvent in a two-phase system; and
e) analyzing the polar fraction by liquid chromatography.

2. The process according to claim 1, wherein
the inoculum is a strain of microalgae of the species Scenedesmus sp. with thermophilic characteristics; and
the culture medium is modified BG-11 culture medium.

3. The process according to claim 2, wherein the modified BG-11 culture medium has urea as the nitrogen source and the trace metal solution is removed from the medium.

4. The process according to claim 1, wherein the flocculation step is carried out via an organic polyamide flocculant at a concentration of 3 mg/L.

5. The process according to claim 1, wherein the extraction step with a biphasic solvent is carried out via a metal/water solvent in a 1:1 ratio and chloroform, forming two fractions that are a polar fraction and a nonpolar fraction.

6. The process according to claim 1, wherein the polar fraction is made up of amino acids, sugars, betaine derivatives, vitamin B3 (nicotinamide or nicotinic acid) and vitamin B5 (pantothenic acid), the monoterpene loliolide and the tryptophan derivative indole-lactic acid.

7. The process according to claim 1, wherein the polar fraction is the biofertilizer/biostimulant.

8. A biofertilizer/biostimulant, obtained by the process as defined in claim 1, comprising amino acids, sugars and betaine derivatives, vitamins B3 and B5, loliolide and indole-lactic acid.

9. A method of direct application of a basic liquid formulation comprising the biofertilizer/biostimulant according to claim 8, wherein the direct application is by farmers and involves making only dilutions.

10. A process of obtaining liquid organomineral fertilizers with additional effects of biofertilizers and biostimulants, wherein the process comprising combining a basic liquid formulation comprising the biofertilizer/biostimulant according to claim 8, with traditional mineral fertilizers with high solubility in water, other soluble fertilizers comprising one or more elements selected from the group consisting of N, P, K, S, Ca, Mg, B, Cu, Zn, Mn, Cl, Mo, and Fe, and other plant nutrients.

11. The process according to claim 10, wherein the traditional mineral fertilizers comprise one or more selected from the group consisting of urea, phosphoric acid, and potassium chloride.

12. A liquid organomineral fertilizer with additional effects of biofertilizers and biostimulants, comprising a basic liquid formulation comprising the biofertilizer/biostimulant according to claim 8, traditional mineral fertilizers with high solubility in water, other soluble fertilizers comprising one or more elements selected from the group consisting of N, P, K, S, Ca, Mg, B, Cu, Zn, Mn, Cl, Mo, and Fe, and other plant nutrients.

13. The liquid organomineral fertilizer according to claim 12, wherein the traditional mineral fertilizers comprise one or more selected from the group consisting of urea, phosphoric acid, and potassium chloride.

14. A process of obtaining a dry organic matrix, wherein the process comprises drying and concentrating the basic liquid formulation comprising the biofertilizer/biostimulant according to claim 8 to obtain the dry organic mix having a moisture content of up to 20% weight/weight.

15. A dry mix obtained by drying and concentrating the basic liquid formulation comprising the biofertilizer/biostimulant according to claim 8, wherein the dry organic mix having a moisture content of up to 20% weight/weight.

16. A process of obtaining liquid organomineral fertilizers with additional effects of biofertilizers and biostimulants, wherein the process comprises drying and concentrating the basic liquid formulation comprising the biofertilizer/biostimulant according to claim 8 to obtain a dry organic mix having a moisture content of up to 20% weight/weight, combining the dry organic mix with other solid traditional mineral fertilizers, other solid fertilizers comprising one or more elements selected from the group consisting of N, P, K, S, Ca, Mg, B, Cu, Zn, Mn, Cl, Mo, Fe, and other mineral nutrients.

17. The process according to claim 16, wherein the traditional mineral fertilizers comprise one or more selected from the group consisting of urea, monoammonium phosphate, diammonium phosphate, and potassium chloride.

18. A method of applying the biofertilizer/biostimulant, obtained by the process as defined in claim 1, wherein the method comprises application of the biofertilizer/biostimulant as an input in agriculture in soybean, corn, sugar cane, cotton and coffee crops, among other cultivated species.

19. The method of claim 15, wherein the application of the biofertilizer/biostimulant is to plants of the Setaria viridis species cultivated in a hydroponic system.
